(19) United States
(12) Patent Application Publication
(10) Pub. No.: US 2016/0199289 A1
(43) Pub. Date: Jul. 14, 2016

(54) COSMETIC ANTIPERSPIRANTS WITH LACTATES

(71) Applicant: Henkel AG & Co. KGaA, Duesseldorf (DE)

(72) Inventors: Bernhard Banowski, Duesseldorf (DE); Marcus Claas, Hilden (DE); Christian Kropf, Hilden (DE)

(73) Assignee: Henkel AG & Co. KGaA, Duesseldorf (DE)

(21) Appl. No.: 15/080,156

(22) Filed: Mar. 24, 2016

Related U.S. Application Data
(63) Continuation of application No. PCT/DE2014/200269, filed on Jun. 18, 2014.

(30) Foreign Application Priority Data
Oct. 15, 2013 (DE) 10 2013 220 779.4

(51) Int. Cl. A61K 8/92; A61Q 15/00; A61K 8/28; A61K 8/26; A61K 8/365

(52) U.S. Cl. CPC A61K 8/92; A61K 8/26; A61K 8/365; A61K 8/28; A61Q 15/00

(57) ABSTRACT

The present invention relates to a cosmetic antiperspirant composition comprising a) at least one substance selected from the group consisting of cosmetic oils which are liquid at 20° C. and 1013 hPa, odorants and waxes, b) at least one antiperspirant aluminum salt in a total amount of 0.1 to 80 wt %, based on the total weight of the cosmetic antiperspirant composition, and c) at least one compound of formula (LA-I), in which R¹ is a linear or branched alkyl group having 1 to 10 carbon atoms, and X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt, the addition of the at least one compound of formula (LA-I) resulting in effective sealing of the efferent ducts of the sweat gland(s).

### FIELD OF THE INVENTION

The present invention generally relates to a cosmetic antiperspirant composition which includes at least one antiperspirant aluminum salt and at least one specific lactate, and wherein the addition of the at least one specific lactate results in an enhancement of the sealing of the sweat gland(s) and/or in a sealing of the sweat gland(s).

The present invention also relates to the use of a combination of at least one antiperspirant aluminum salt and at least one specific lactate to reduce and/or prevent underarm perspiration.

The present invention additionally relates to a non-therapeutic cosmetic method for preventing and/or reducing the perspiration of the body, in which the antiperspirant composition according to the invention is applied to the skin, in particular to the skin of the armpits.

The present invention finally relates to the use of a specific lactate for enhancing the sealing of the sweat gland(s) and/or for sealing the sweat gland(s).

### BACKGROUND OF THE INVENTION

The washing, cleaning and care of one's body is a basic human need, and modern industry is constantly attempting to meet these human needs in many ways. The lasting elimination or at least reduction in body odor and underarm wetness is particularly important for daily hygiene. In the prior art, numerous specific deodorizing or antiperspirant body care compositions are known which have been developed for use in body regions having a high density of sweat glands, in particular in the underarm area. These are packaged in various administration forms, for example as a powder, in stick form, as an aerosol spray, pump spray, liquid and gel-like roll-on application, cream, gel and as impregnated flexible substrates (deodorant wipes).

Cosmetic antiperspirants of the prior art include at least one antiperspirant salt in addition to at least one oil or one fatty substance and an odorant component or a fragrance.

The antiperspirant salts used in antiperspirants reduce on the one hand the sweat secretion of the body by a temporary narrowing or blocking of the efferent ducts of the sweat glands, so that the amount of sweat can be reduced by around 20 to 60 percent. On the other hand, due to their antimicrobial action, they have an additional deodorizing effect.

As the antiperspirant salt, use is usually made of activated basic aluminum and aluminum zirconium halides, as described in the documents EP 0308937 A2, EP 0183171 A2, U.S. Pat. No. 4,359,456 A and EP 0191628. Use may also be made of aluminum and aluminum zirconium halides which are stabilized with organic salts as complex ligands, as disclosed for example in the documents U.S. Pat. No. 3,542,919 A, U.S. Pat. No. 3,553,316 A, U.S. Pat. No. 3,991,176 A and WO 2005/092795 A1.

One disadvantage of the aforementioned activated basic aluminum and aluminum zirconium halides of the prior art lies in the changing of the polymer structure in aqueous solution as the storage time increases and/or when using protic solvents or a limited effect of non-activated, inexpensive aluminum salts, so that a significant reduction in sweat secretion of the body due to the temporary narrowing and/or blocking of the efferent ducts of the sweat glands cannot always be ensured.

There is therefore a need for cosmetic antiperspirant compositions which do not exhibit any significant reduction in the antiperspirant effect even during long storage times and/or when using high quantities of protic solvents. The antiperspirants should also be able to be produced inexpensively.

The object of the present invention was to provide a cosmetic antiperspirant composition which avoids or at least alleviates the disadvantages of the prior art and which ensures a reliable blocking of the efferent ducts of the sweat glands even during relatively long storage times and/or in the presence of protic solvents.

It has now surprisingly been found that the use of specific lactates in cosmetic compositions which include antiperspirant aluminum salts leads to a significantly increased hydrogel formation of these compositions at pH values which prevail exclusively within the efferent ducts of the sweat glands, and thus ensures an effective blocking of these efferent ducts. Due to the pH selectivity, a hydrogel formation does not take place either in the cosmetic antiperspirant composition or on the outside of the skin, so that no reduced efficacy of the cosmetic antiperspirant composition due to premature undesired hydrogel formation occurs as a result of adding the specific lactates.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic antiperspirant composition comprising at least one substance selected from the group consisting of cosmetic oils which are liquid at 20° C. and 1013 hPa, odorants and waxes; at least one antiperspirant aluminum salt in a total amount of 0.1 to 80 wt %, based on the total weight of the cosmetic antiperspirant composition; and at least one compound of formula (LA-I),

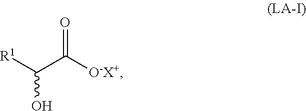

in which R¹ is a linear or branched alkyl group having 1 to 10 carbon atoms, and X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt.

The use of a combination of at least one substance selected from the group consisting of cosmetic oils which are liquid at 20° C. and 1013 hPa, odorants and waxes, at least one antiperspirant aluminum salt in a total amount of 0.1 to 80 wt %, based on the total weight of the combination, and at least one compound of formula (LA-I)

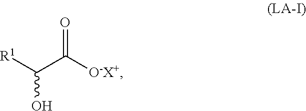

in which R¹ is a linear or branched alkyl group having 1 to 10 carbon atoms, and X⁺ is Li⁺, K⁺, NH₄⁺, ½ Mg²⁺, ½ Mn²⁺, ½ Zn²⁺, ⅓ Al³⁺, ¼ Zr⁴⁺ or at least one antiperspirant aluminum salt, for reducing and/or preventing sweat.

A non-therapeutic cosmetic method for preventing and/or reducing the perspiration of the body, in which a cosmetic antiperspirant composition, comprising at least one substance selected from the group consisting of cosmetic oils which are liquid at 20° C. and 1013 hPa, odorants and waxes, at least one antiperspirant aluminum salt in a total amount of 0.1 to 80 wt %, based on the total weight of the cosmetic antiperspirant composition, and at least one compound of formula (LA-I), is applied to the skin, in particular to the skin of the armpits.

The use of at least one compound of formula (LA-I)

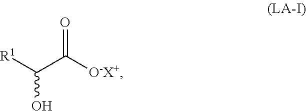

in which R¹ is a linear or branched alkyl group having 1 to 10 carbon atoms, and X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt, for enhancing the sealing of the sweat gland(s) and/or for sealing the sweat gland(s).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is a cosmetic antiperspirant composition comprising
a) at least one substance selected from the group consisting of cosmetic oils which are liquid at 20° C. and 1013 hPa, odorants and waxes,
b) at least one antiperspirant aluminum salt in a total amount of 0.1 to 80 wt %, based on the total weight of the cosmetic antiperspirant composition, and
c) at least one compound of formula (LA-I),

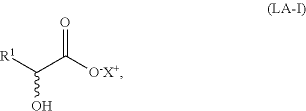

in which
R¹ is a linear or branched alkyl group having 1 to 10 carbon atoms, and
X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt.

By combining antiperspirant aluminum salts with specific compounds of formula (LA-I), a hydrogel formation of the antiperspirant aluminum salt and of the compound of formula (LA-I) takes place exclusively at extremely specific pH values, without wishing to be restricted to this theory. Due to the extremely selective formation of the hydrogel at pH values which exist only within the efferent ducts of the sweat glands, an effective blocking of the efferent ducts of the sweat glands is ensured without the antiperspirant effect of the cosmetic composition according to the invention being reduced due to premature undesired hydrogel formation as a result of adding the compound of formula (LA-I). An excellent antiperspirant effect can thus be ensured even after relatively long storage times and/or in the presence of high quantities of protic solvents.

According to the invention, the term “antiperspirant” will be understood to mean a decrease or reduction in perspiration of the sweat glands of the body.

Furthermore, within the context of the present invention, the term “cosmetic oil” will be understood to mean an oil which is suitable for cosmetic use and which is immiscible with water. Furthermore, the cosmetic oil used according to the invention is neither an odorant nor an essential oil.

Moreover, within the context of the present invention, the term “odorants” will be understood to mean substances having a molar mass of 74 to 300 g/mol which include at least one osmophoric group in the molecule and which have an odor and/or taste, that is to say they are capable of stimulating the receptors of the hair cells of the olfactory system. Osmophoric groups are groups covalently bonded to the molecule backbone, in the form of hydroxyl groups, formyl groups, oxo groups, alkoxycarbonyl groups, nitrile groups, nitro groups, azide groups, etc. In this connection, fragrance oils which are liquid at 20° C. and 1013 hPa, fragrances or fragrance oil constituents also fall under the term “odorants” within the context of the present invention.

In addition, within the context of the present invention, the term “waxes” will be understood to mean substances which at 20° C. are kneadable or solid to brittle and hard, have a coarse to fine crystalline structure and are colored and translucent to opaque, but not vitreous. Furthermore, these substances melt above 25° C. without decomposing, are slightly liquid (less viscous) a little above the melting point, have a greatly temperature-dependent consistency and solubility, and can be polished under slight pressure.

According to the invention, the term “volatile cosmetic oil” refers to cosmetic oils which, at 20° C. and an ambient pressure of 1013 hPa, have a vapor pressure of 2.66 Pa to 40,000 Pa (0.02 to 300 mm Hg), preferably 10 to 12,000 Pa (0.1 to 90 mm Hg), more preferably 13 to 3000 Pa (0.1 to 23 mm Hg), in particular 15 to 500 Pa (0.1 to 4 mm Hg).

Moreover, within the context of the present invention, the term “non-volatile cosmetic oils” will be understood to mean cosmetic oils which, at 20° C. and an ambient pressure of 1013 hPa, have a vapor pressure of less than 2.66 Pa (0.02 mm Hg).

Furthermore, the term “fatty acid,” as used in the context of the present invention, will be understood to mean aliphatic carboxylic acids which have unbranched or branched carbon radicals having 4 to 40 carbon atoms. The fatty acids used in the context of the present invention may be both naturally occurring and synthetically produced fatty acids. Furthermore, the fatty acids may be monounsaturated or polyunsaturated.

Finally, within the context of the present invention, the term “fatty alcohol” will be understood to mean aliphatic, monovalent, primary alcohols which have unbranched or branched hydrocarbon radicals having 4 to 40 carbon atoms. The fatty alcohols used in the context of the present invention may also be monounsaturated or polyunsaturated.

In the present case, unless indicated otherwise, the specification wt % refers to the total weight of the cosmetic antiperspirant composition according to the invention without any propellant that may be present.

According to one preferred embodiment of the present invention, the cosmetic oil which is liquid at 20° C. and 1013 hPa is selected from the group consisting of

(i) volatile cyclic silicone oils, in particular cyclotrisiloxane, cyclotetrasiloxane, cyclopentasiloxane and cyclohexasiloxane, and linear silicone oils having 2 to 10 siloxane units, in particular hexamethyldisiloxane, octamethyltrisiloxane, decamethyltetrasiloxane;
(ii) volatile non-silicone oils, in particular liquid paraffin oils and isoparaffin oils, such as isodecane, isoundecane, isododecane, isotridecane, isotetradecane, isopentadecane, isohexadecane and isoeicosane;
(iii) non-volatile silicone oils, in particular linear polyalkylsiloxanes of higher molecular weight;
(iv) non-volatile non-silicone oils, in particular the esters of linear or branched, saturated or unsaturated C₂₋₃₀ fatty alcohols with linear or branched, saturated or unsaturated C₂₋₃₀ fatty acids, which may be hydroxylated, the C₈-C₂₂ fatty alcohol esters of monovalent or polyvalent C₂-C₇ hydroxycarboxylic acids, the triethyl citrates, the branched, saturated or unsaturated C₆₋₃₀ fatty alcohols, the mono-, di- and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide to monovalent or polyvalent C₃₋₂₂ alkanols, which may optionally be esterified, the symmetrical, asymmetrical or cyclic esters of carbonic acid with fatty alcohols, the esters of dimers of unsaturated C₁₂₋₂₂ fatty acids with monovalent, linear, branched and cyclic C₂₋₁₈ alkanols or C₂₋₆ alkanols, the benzoic acid esters of linear or branched C₈₋₂₂ alkanols, such as benzoic acid C₁₂₋₁₅ alkyl ester and benzoic acid isostearyl ester and benzoic acid octyl dodecyl ester, the synthetic hydrocarbons, such as polyisobutene and polydecene, the alicyclic hydrocarbons; and
(v) mixtures thereof.

The use of volatile silicone oils and volatile non-silicone oils in the cosmetic antiperspirant compositions according to the invention results in a drier skin feel and in a faster release of the antiperspirant aluminum salt.

The cyclic volatile silicone oils which can be used in the context of the invention have a vapor pressure of 13 to 15 Pa (0.1 mm Hg) at 20° C. and an ambient pressure of 1013 hPa. Furthermore, according to the invention, a low-molecular-weight phenyl trimethicone having a vapor pressure of around 2000 Pa (15 mm Hg) at 20° C. and an ambient pressure of 1013 hPa can also be used as a linear volatile silicone oil. Due to the high persistence of cyclodimethicone in the environment, however, it may be preferred according to the invention if 0 to less than 1 wt %, preferably 0 to less than 0.1 wt %, cyclic volatile silicone oils are used in the cosmetic antiperspirant compositions according to the invention.

According to the invention, use is preferably made of volatile non-silicone oils in the form of C₁₀₋₁₃ isoparaffin mixtures having a vapor pressure of 10 to 400 Pa (0.08 to 3 mm Hg), preferably 13 to 100 Pa (0.1 to 0.8 mm Hg), at 20° C. and an ambient pressure of 1013 hPa. In this case, it is preferred in the context of the present invention if the volatile C₈-C₁₆ isoparaffin is included in a total amount of 1 to 60 wt %, preferably 3 to 45 wt %, preferably 5 to 40 wt %, in particular 8 to 20 wt %, based on the total weight of the cosmetic antiperspirant composition. Of course, it is likewise possible to formulate cosmetic antiperspirant compositions according to the invention with a lesser proportion of volatile oils—that is to say with 0.5 to 15 wt % volatile oils, based on the total weight of the cosmetic antiperspirant composition—or even without volatile oils.

In order to mask insoluble constituents, such as talc or antiperspirant aluminum salts that have dried onto the skin, it may be preferred according to the invention if the cosmetic antiperspirant compositions include a non-volatile silicone oil and/or a non-volatile non-silicone oil.

In this connection, cosmetic antiperspirant compositions which are preferred according to the invention include at least one ester of linear or branched, saturated or unsaturated fatty alcohols having 2 to 30 carbon atoms with linear or branched, saturated or unsaturated fatty acids having 2 to 30 carbon atoms, which may be hydroxylated, in a total amount of 1 to 30 wt %, preferably 5 to 26 wt %, preferably 9 to 24 wt %, in particular 12 to 17 wt %, based on the total weight of the cosmetic antiperspirant composition.

Within the context of the present invention, linear polyalkylsiloxanes having a kinematic viscosity at 25° C. of 5 to 2000 cSt, in particular linear polydimethylsiloxanes having a kinematic viscosity at 25° C. of 5 to 2000 cSt, preferably 10 to 350 cSt, in particular 50 to 100 cSt, may be used as non-volatile silicone oils. The aforementioned non-volatile silicone oils are obtainable under the trade name Dow Corning® 200 or Xiameter PMX from Dow Corning or Xiameter. Other preferred non-volatile silicone oils are phenyltrimethicones having a kinematic viscosity at 25° C. of 10 to 100 cSt, preferably 15 to 30 cSt, and cetyldimethicones.

Also preferred according to the invention is the use of mixtures of the aforementioned cosmetic oils, in particular of non-volatile and volatile cosmetic oils, since in this way parameters such as skin feel, visibility of residue and stability of the cosmetic antiperspirant composition according to the invention can be adjusted and the composition can thus be better adapted to the requirements of the consumers.

Within the context of the present invention, it is preferred if the cosmetic oil which is liquid at 20° C. and 1013 hPa is included in a total amount of 1 to 95 wt %, preferably 10 to 85 wt %, preferably 20 to 75 wt %, more preferably 35 to 70 wt %, in particular 50 to 60 wt %, based on the total weight of the cosmetic antiperspirant composition.

However, it may also be preferred if the cosmetic oil which is liquid at 20° C. and 1013 hPa is included in a total amount of 0.2 to 70 wt %, preferably 2 to 60 wt %, preferably 3 to 50 wt %, more preferably 5 to 35 wt %, in particular 8 to 20 wt %, based on the total weight of the cosmetic antiperspirant composition. In this connection, it may also be provided according to the invention that the antiperspirant compositions according to the invention include less than 0.2 wt %, preferably less than 0.1 wt %, in particular 0 wt % of the cosmetic oil which is liquid at 20° C. and 1013 hPa. The use of only extremely small amounts of the cosmetic oil which is liquid at 20° C. and 1013 hPa is particularly preferred in cosmetic antiperspirant compositions according to the invention which are in aqueous, aqueous-alcoholic or aqueous-glycolic form.

According to one preferred embodiment of the present invention, the odorant is selected from the group consisting of

(i) esters, in particular benzyl acetate, phenoxyethyl isobutyrate, p-tert.-butylcyclohexyl acetate, linalyl acetate, dimethyl benzyl carbinyl acetate (DMBCA), phenylethyl acetate, benzyl acetate, ethyl methyl phenyl glycinate, allyl cyclohexyl propionate, styrallyl propionate, benzyl salicylate, cyclohexyl salicylate, Floramat, Melusat and Jasmecyclat;
(ii) ethers, in particular benzyl ethyl ether and Ambroxan;
(iii) aldehydes, in particular linear alkanals having 8 to 18 carbon atoms, citral, citronellal, citronellyloxyacetaldehyde, cyclamen aldehyde, lilial and bourgeonal;
(iv) ketones, in particular ionone, alpha-isomethyl ionone and methyl cedryl ketone;
(v) alcohols, in particular anethol, citronellol, eugenol, geraniol, linalool, phenylethyl alcohol and terpineol;
(vi) hydrocarbons, in particular terpenes such as limonene and pinene; and
(vii) mixtures thereof.

However, use is preferably made of mixtures of different odorants which together produce a pleasant scent.

Particularly pleasantly smelling cosmetic antiperspirant compositions according to the invention will be obtained if the odorant is included in a total amount of 0.00001 to 10 wt %, preferably 0.001 to 9 wt %, preferably 0.01 to 8 wt %, more preferably 0.5 to 7 wt %, in particular 1 to 6 wt %, based on the total weight of the cosmetic antiperspirant composition. In this connection, however, it may also be provided that the odorant is included in a total amount of 0.00001 to 5 wt %, preferably 0.001 to 4 wt %, preferably 0.01 to 3 wt %, more preferably 0.1 to 2 wt %, in particular 0.2 to 1.5 wt %, based on the total weight of the propellant-containing cosmetic antiperspirant composition.

According to a further preferred embodiment of the present invention, the wax is selected from the group consisting of

(i) fatty acid glycerol mono-, di- and triesters;
(ii) Butyrospermum Parkii (shea butter);
(iii) esters of saturated, monovalent C₈₋₁₈ alcohols with saturated C₁₂₋₁₈ monocarboxylic acids;
(iv) linear, primary C₁₂-C₂₄ alkanols;
(v) esters of a saturated, monovalent C₁₆-C₆₀ alkanol and a saturated C₈-C₃₆ monocarboxylic acid, in particular cetyl behenate, stearyl behenate and C₂₀-C₄₀ alkyl stearate;
(vi) glycerol triesters of saturated linear C₁₂-C₃₀ carboxylic acids, which may be hydroxylated, in particular hydrogenated palm oil, hydrogenated coconut oil, hydrogenated castor oil, glyceryl tribehenate and glyceryl tri-12-hydroxystearate;
(vii) natural vegetable waxes, in particular candelilla wax, carnauba wax, Japan wax, sugarcane wax, ouricury wax, cork wax, sunflower wax, fruit waxes;
(viii) animal waxes, in particular beeswax, shellac wax and spermaceti;
(ix) synthetic waxes, in particular montan waxes, hydrogenated jojoba waxes and Sasol waxes, polyalkylene waxes and polyethylene glycol waxes, C₂₀-C₄₀ dialkyl esters of dimeric acids, C₃₀₋₅₀ alkyl beeswax and alkyl and alkylaryl esters of dimer fatty acids, paraffin waxes; and
(x) mixtures thereof.

Particular preference is given to commercial products having the INCI name Cocoglycerides, in particular the commercial products Novata® (ex BASF), particularly preferably Novata® AB, a mixture of C₁₂₋₁₈ mono-, di- and triglycerides which melts in the range from 30 to 32° C., and the products of the Softisan range (Sasol Germany GmbH) having the INCI name Hydrogenated Cocoglycerides, in particular Softisan 100, 133, 134, 138, 142. Other preferred esters of saturated, monovalent C₁₂₋₁₈ alcohols with saturated C₁₂₋₁₈ monocarboxylic acids are stearyl laurate, cetearyl stearate (for example Crodamol® CSS), cetyl palmitate (for example Cutina® CP) and myristyl myristate (for example Cetiol® MM). Furthermore, a C₂₀-C₄₀ alkyl stearate is preferably used as the wax component. This ester is known under the name Kesterwachs® K82H or Kesterwachs® K80H and is sold by Koster Keunen Inc.

Within the context of the present invention, it is preferred if the wax is included in a total amount of 0.01 to 20 wt %, preferably 3 to 20 wt %, preferably 5 to 18 wt %, in particular 6 to 15 wt %, based on the total weight of the cosmetic antiperspirant composition.

Within the context of the present invention, a particularly good antiperspirant effect is obtained if the antiperspirant aluminum salt is included in a total amount of 1 to 40 wt %, preferably 3 to 35 wt %, preferably 4 to 32 wt %, more preferably 5 to 28 wt %, even more preferably 8 to 25 wt %, in particular 12 to 22 wt %, based on the total weight of the cosmetic antiperspirant composition. However, it may also be provided that the antiperspirant aluminum salt is included in a total amount of 0.1 to 20 wt %, preferably 0.5 to 15 wt %, preferably 1 to 12 wt %, more preferably 1.5 to 10 wt %, in particular 2 to 8 wt %, based on the total weight of the propellant-containing antiperspirant composition.

Within the context of the present invention, the antiperspirant aluminum salt may be selected from the group consisting of

(i) water-soluble astringent inorganic salts of aluminum, in particular aluminum chlorohydrate, aluminum sesquichlorohydrate, aluminum dichlorohydrate, aluminum hydroxide, potassium aluminum sulphate, aluminum bromohydrate, aluminum chloride, aluminum sulphate;
(ii) water-soluble astringent organic salts of aluminum, in particular aluminum chlorohydrex propylene glycol, aluminum chlorohydrex polyethylene glycol, aluminum-propylene glycol complexes, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate;
(iii) water-soluble astringent inorganic aluminum zirconium salts, in particular aluminum zirconium trichlorohydrate, aluminum zirconium tetrachlorohydrate, aluminum zirconium pentachlorohydrate, aluminum zirconium octachlorohydrate;
(iv) water-soluble astringent organic aluminum zirconium salts, in particular aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine; and
(v) mixtures thereof.

According to the invention, aluminosilicates and zeolites do not fall under the term “antiperspirant aluminum salts.” Furthermore, according to the invention, water-soluble salts will be understood to mean those salts which have a solubility of at least 3 wt % at 20° C., that is to say at least 3 g of the antiperspirant aluminum salt dissolve in 97 g of water at 20° C.

Particularly preferred inorganic aluminum salts are selected from aluminum chlorohydrate, in particular aluminum chlorohydrate having the general formula [Al₂(OH)₅Cl.1-6H₂O]ₙ, preferably [Al₂(OH)₅Cl.2-3H₂O]ₙ, which may be in non-activated (polymerized) or in activated (depolymerized) form, and aluminum chlorohydrate having the general formula [Al₂(OH)₄Cl₂.1-6H₂O]ₙ, preferably [Al₂(OH)₄Cl₂.2-3H₂O]ₙ, which may be in non-activated (polymerized) or in activated (depolymerized) form. The production of such antiperspirant aluminum salts is disclosed for example in the documents U.S. Pat. No. 3,887,692 A, U.S. Pat. No. 3,904,741 A, U.S. Pat. No. 4,359,456 A, GB 2048229 A and GB 1347950 A.

Antiperspirant aluminum salts which are particularly preferred according to the invention are selected from so-called “activated” aluminum salts, which are also known as antiperspirant active ingredients “with enhanced activity.” Such active ingredients are known in the prior art and are also commercially available. The production thereof is disclosed for example in the documents GB 2048229 A, U.S. Pat. No. 4,775,528 A and U.S. Pat. No. 6,010,688 A. Activated aluminum salts are usually produced by heat treatment of a dilute solution of the salt in question (for example a solution containing 10 wt % salt), in order to increase the HPLC peak 4 to peak 3 area ratio thereof. The activated salt can then be dried to a powder, in particular by spray drying. Besides spray drying, drum drying is also suitable for example. Activated aluminum salts typically have an HPLC peak 4 to peak 3 area ratio of at least 0.4, preferably at least 0.7, in particular at least 0.9, wherein at least 70% of the aluminum can be assigned to these HPLC peaks.

In this connection, “activated” aluminum zirconium salts are also known, which have a high HPLC peak 5 aluminum content, in particular a peak 5 area of at least 33%, preferably at least 45%, based on the total area below peaks 2 to 5, measured by HPLC of a 10 wt % strength aqueous solution of the active ingredient under conditions at which the aluminum species are resolved into at least 4 successive peaks (known as peaks 2 to 5). Preferred aluminum zirconium salts having a high HPLC peak 5 aluminum content (also known as “E⁵ AZCH”) are disclosed for example in the documents U.S. Pat. No. 6,436,381 A and U.S. Pat. No. 6,649,152 A. Furthermore, the aforementioned activated aluminum zirconium salts can additionally be stabilized with a water-soluble strontium salt and/or with a water-soluble calcium salt, as disclosed for example in the document U.S. Pat. No. 6,923,952 A.

According to the invention, it is also possible to use antiperspirant aluminum salts as non-aqueous solutions or solubilisates of an activated antiperspirant aluminum or aluminum zirconium salt, for example according to the document U.S. Pat. No. 6,010,688 A. Such aluminum or aluminum zirconium salts are stabilized against the loss of activation of the salt by adding an effective amount of a polyvalent alcohol which has 3 to 6 carbon atoms and 3 to 6 hydroxyl groups, preferably propylene glycol, sorbitol and pentaerythritol.

Particular preference is also given to complexes of activated antiperspirant aluminum or aluminum zirconium salts with a polyvalent alcohol which include 20 to 50 wt %, preferably 20 to 42 wt %, activated antiperspirant aluminum or aluminum zirconium salt and 2 to 16 wt % molecularly bound water, wherein the remainder up to 100 wt % is at least one polyvalent alcohol having 3 to 6 carbon atoms and 3 to 6 hydroxyl groups. Propylene glycol, propylene glycol/sorbitol mixtures and propylene glycol/pentaerythritol mixtures are preferred alcohols of this type. Such complexes of an activated antiperspirant aluminum or aluminum zirconium salt with a polyvalent alcohol, which are preferred according to the invention, are disclosed for example in the documents U.S. Pat. No. 5,643,558 A and U.S. Pat. No. 6,245,325 A.

Within the context of the present invention, it is likewise possible to use basic calcium aluminum salts, as disclosed for example in the document U.S. Pat. No. 2,571,030 A, as antiperspirant aluminum salts. These salts can be obtained by reacting calcium carbonate with aluminum chlorhydroxide or aluminum chloride and aluminum powder or by adding calcium chloride dihydrate to aluminum chlorhydroxide. However, it is also possible to use aluminum-zirconium complexes which are buffered with salts of amino acids, in particular with alkali and alkaline earth metal glycinates, as disclosed for example in the document U.S. Pat. No. 4,017,599 A.

Other preferred activated aluminum salts are those of general formula Al₂(OH)₆₋ₐXa, in which X is Cl, Br, I or NO₃ and “a” is a number from 0.3 to 5, preferably from 0.8 to 2.5, in particular from 1 to 2, so that the molar ratio of Al:X is 0.9:1 to 2.1:1. Such activated antiperspirant aluminum salts are disclosed for example in the document U.S. Pat. No. 6,074,632 A. Particular preference is given to aluminum chlorohydrate (i.e. X in the aforementioned formula is Cl) and specifically to 5/6-basic aluminum chlorohydrate where “a”=1, so that the molar ratio of aluminum to chlorine is 1.9:1 to 2.1:1.

Preferred activated aluminum zirconium salts are those of general formula ZrO(OH)_{2-pb}Y_{b}, in which Y is Cl, Br, I, NO₃ or SO₄, b is a rational number from 0.8 to 2 and p is the valency of Y, so that the Al:Zr molar ratio is 2 to 10 and the metal:(X+Y) ratio is 0.73 to 2.1, preferably 0.9 to 1.5. Such activated antiperspirant aluminum zirconium salts are disclosed for example in the aforementioned document U.S. Pat. No. 6,074,632 A. One particularly preferred salt is aluminum zirconium chlorohydrate (i.e. X and Y are Cl), which has an Al:Zr ratio of 2 to 10 and a molar metal:Cl ratio of 0.9 to 2.1. Preferred antiperspirant active ingredients are disclosed in the documents U.S. Pat. No. 6,663,854 A and US 2004/0009133 A1.

Antiperspirant aluminum salts which are particularly preferred according to the invention have a molar metal-to-chloride ratio of 1.9 to 2.1. The metal-to-chloride ratio of aluminum sesquichlorohydrates, which are likewise particularly preferred in the context of the invention, is 1.5:1 to 1.8:1. Preferred aluminum zirconium tetrachlorohydrates have a molar ratio of Al:Zr of 2 to 6 and of metal:chloride of 0.9 to 1.3, preference being given in particular to salts having a molar metal-to-chloride ratio of 0.9 to 1.1, preferably 0.9 to 1.0.

Within the context of the present invention, a particularly effective hydrogel formation between the compound of formula (LA-I) and the antiperspirant aluminum salts is achieved if the compound of formula (LA-I) is included in a total amount of 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %, based on the total weight of the cosmetic antiperspirant composition. Furthermore, it may be provided in the context of the present invention that the compound of formula (LA-I) is included in a total amount of 0.05 to 40 wt %, preferably 0.5 to 36 wt %, preferably 1 to 31 wt %, more preferably 3 to 29 wt %, even more preferably 5 to 27 wt %, in particular 8 to 25 wt %, based on the total weight of the cosmetic antiperspirant composition. However, it is also possible in the context of the invention that the cosmetic antiperspirant compositions include the compound of formula (LA-I) in a total amount of 0.05 to 40 wt %, preferably 0.3 to 36 wt %, preferably 0.5 to 30 wt %, more preferably 0.7 to 20 wt %, even more preferably 1.0 to 18 wt %, in particular 1.5 to 12 wt %, based on the total weight of the propellant-containing cosmetic antiperspirant composition. Without wishing to be restricted to this theory, the use of the aforementioned amount(s) of the compound of formula (LA-I) results in a significantly increased and extremely pH-selective hydrogel formation with the antiperspirant aluminum salt. In this way, an effective blocking of the efferent ducts of the sweat glands, and thus an excellent antiperspirant effect, is ensured. Furthermore, the use of the aforementioned amounts of the compound of formula (LA-I) does not lead to an undesired premature hydrogel formation in the cosmetic antiperspirant composition or on the skin surface, so that the excellent antiperspirant effect is ensured even over long storage times.

Particularly good results in terms of blocking the efferent ducts of the sweat glands through hydrogel formation are obtained if the cosmetic antiperspirant composition includes at least one compound of formula (LA-Ia) and/or (LA-Ib)

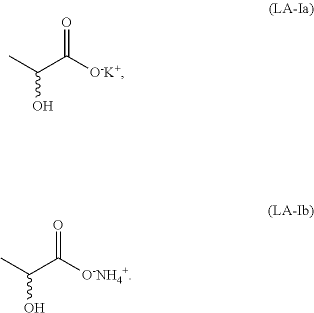

The combination or mixture of the potassium salt of lactic acid (formula LA-Ia) and/or of the ammonium salt of lactic acid (formula LA-Ib) with at least one antiperspirant aluminum salt results in an extremely pH-selective enhanced hydrogel formation at pH values which exist within the efferent ducts of the sweat glands, so that an effective blocking of these efferent ducts by virtue of hydrogel formation, and thus an excellent antiperspirant effect, is ensured. In contrast, the use of the sodium salt of lactic acid does not lead to hydrogel formation at pH values which exist within the efferent ducts of the sweat glands. Thus, only the use of specific lactates of formula (LA-I) in the cosmetic antiperspirant compositions according to the invention results in a significant hydrogel formation and thus in an effective blocking of the efferent ducts of the sweat glands.

The antiperspirant effect of the cosmetic antiperspirant compositions according to the invention can be further improved if the cosmetic antiperspirant composition has a weight ratio of the antiperspirant aluminum salt to the compound of formula (LA-I) of 40:1 to 19:1, preferably 30:1 to 16:1, preferably 20:1 to 15:1, more preferably 10:1 to 13:1, even more preferably 9:1 to 12:1, in particular 8:1 to 1:1. In this connection, it may also be provided that the cosmetic antiperspirant composition has a weight ratio of the antiperspirant aluminum salt to the compound of formula (LA-I) of 1:1.5 to 1:40, preferably 1:2 to 1:35, more preferably 1:2.5 to 1:30, even more preferably 1:3 to 1:25, in particular 1:3.5 to 1:20. The aforementioned weight ratio refers to the total amount of all antiperspirant aluminum salts and to the total amount of all compounds of formula (LA-I) in the cosmetic antiperspirant composition.

Within the context of one particularly preferred embodiment, the weight ratio of the antiperspirant aluminum salt to the compound of formula (LA-I) is 1.5:1. In addition, the use of a weight ratio of the antiperspirant aluminum salt to the compound of formula (LA-I) of 10:1 also leads to cosmetic antiperspirant compositions according to the invention which have an excellent antiperspirant effect. Furthermore, when using the aforementioned weight ratio, use may even be made of protic solvents, particularly in high concentrations or amounts, without this having any negative effect on the antiperspirant effect of the cosmetic antiperspirant compositions according to the invention, so that an extremely flexible packaging of the compositions according to the invention is possible.

According to a further embodiment of the present invention, the cosmetic antiperspirant composition includes no zirconium-containing compounds. The avoidance, according to the invention, of the use of zirconium-containing antiperspirant compounds, such as zirconium aluminum mixed salts, results in the cosmetic antiperspirant compositions according to the invention being cheaper to produce, since the raw ingredients for producing the zirconium-containing compounds are more expensive.

The cosmetic antiperspirant composition preferably includes free water in a total amount of less than 10 wt %, preferably less than 8 wt %, preferably less than 5 wt %, more preferably less than 3 wt %, even more preferably less than 1 wt %, in particular 0 wt %, based on the total weight of the cosmetic antiperspirant composition. Within the context of the present invention, free water will thus be understood to mean water which is different from water of crystallization, hydration water or similarly molecularly bound water of the constituents used, in particular of the antiperspirant aluminum salts.

It has surprisingly been found that the blocking of the efferent ducts of the sweat glands by hydrogel formation, which takes place when at least one antiperspirant aluminum salt is combined with at least one compound of formula (LA-I), can be significantly increased if the cosmetic antiperspirant compositions according to the invention include free water in an amount of 15 to 96 wt %, based on the total weight of the cosmetic antiperspirant composition. In one particularly preferred embodiment of the present invention, the cosmetic antiperspirant composition therefore includes free water in a total amount of 15 to 96 wt %, preferably 25 to 80 wt %, preferably 30 to 70 wt %, in particular 40 to 60 wt %, based on the total weight of the cosmetic antiperspirant composition.

The cosmetic antiperspirant compositions according to the invention preferably exist as a suspension of the undissolved antiperspirant aluminum salt in the cosmetic oil which is liquid at 20° C. and 1013 hPa.

In another preferred administration form, the cosmetic antiperspirant composition exists as a water-in-oil emulsion. In this case, it may in particular be a sprayable water-in-oil emulsion, which can be sprayed by means of a propellant. In this connection, it is preferred if the cosmetic antiperspirant composition according to the invention which exists in the form of a water-in-oil emulsion includes the compound of formula (LA-I) in a total amount of 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %, based on the total weight of the cosmetic antiperspirant composition.

Within the context of the present invention, it may also be provided that the cosmetic antiperspirant composition exists as an oil-in-water emulsion. In this case, the composition according to the invention is preferably sprayed as a propellant-free pump spray or squeeze spray or applied as a roll-on. In this connection, it is preferred if the cosmetic antiperspirant composition which exists in the form of an oil-in-water emulsion includes the compound of formula (LA-I) in a total amount of 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %, based on the total weight of the cosmetic antiperspirant composition.

It may also be provided according to the invention that the cosmetic antiperspirant composition exists as an aqueous, aqueous-alcoholic or aqueous-glycolic solution. Due to the combination according to the invention of an antiperspirant aluminum salt with at least one compound of formula (LA-I), even protic solvents, such as aqueous solutions, can be used to formulate the cosmetic antiperspirant compositions according to the invention, without any significant reduction in the antiperspirant effect occurring. The addition of the at least one compound of formula (LA-I) ensures an effective sealing of the efferent ducts of the sweat glands and thus an excellent antiperspirant effect, even when protic solvents are used.

According to one preferred embodiment of the present invention, the cosmetic antiperspirant composition includes ethanol in a total amount of 1 to 50 wt %, preferably 5 to 40 wt %, preferably 7 to 35 wt %, in particular 10 to 30 wt %, based on the total weight of the cosmetic antiperspirant composition. However, it may also be provided that the cosmetic antiperspirant composition includes ethanol in a total amount of 10 to 95 wt %, preferably 15 to 90 wt %, preferably 20 to 87 wt %, more preferably 30 to 85 wt %, in particular 40 to 80 wt %, based on the total weight of the cosmetic antiperspirant composition. As mentioned above, due to the use of the compound of formula (LA-I), even large amounts of protic solvents, such as ethanol, can be used without this having any negative effect on the antiperspirant effect of the cosmetic antiperspirant composition according to the invention.

The cosmetic antiperspirant composition according to the invention can be applied by various methods. According to one preferred embodiment, the cosmetic antiperspirant composition is packaged as a spray application. The spray application takes place using a spray device which includes in a container a filling of the cosmetic antiperspirant composition according to the invention in liquid, viscous-flowable, suspension or powder form. The filling may be under the pressure of a propellant (compressed gas cans, compressed gas packagings, aerosol packagings), or it may be a mechanically operated pump atomizer without a propellant gas (pump sprays/squeeze bottles). The containers have a withdrawal device, preferably in the form of valves, which enable the withdrawal of the contents as a mist, puff, foam, powder, paste or liquid jet. Especially suitable containers for the spray devices are cylindrical vessels made of metal (aluminum, tinplate, capacity preferably at most 1000 ml), protected and/or non-splintering glass or plastic (capacity preferably at most 220 ml) or splintering glass or plastic (capacity preferably 50 to 400 ml). Compositions in cream, gel, paste and liquid form may be packaged for example in pump, spray or squeeze dispensers, in particular also in multi-chamber pump dispensers, multi-chamber spray dispensers or multi-chamber squeeze dispensers. The packaging for the compositions according to the invention may be opaque, but may also be transparent or translucent.

The cosmetic antiperspirant composition is preferably packaged as a stick, soft solid, cream, roll-on, dibenzylidene alditol-based gel, loose or compact powder. The formulation of the cosmetic antiperspirant compositions according to the invention in a particular administration form, such as for example an antiperspirant roll-on, an antiperspirant stick or an antiperspirant gel, preferably depends on the requirements of the intended use. Depending on the intended use, therefore, the cosmetic antiperspirant compositions according to the invention may exist in solid, semi-solid, liquid, disperse, emulsified, suspended, gel-like, multiphase or powder form. Within the context of the present invention, the term liquid also encompasses any types of solids dispersions in liquids. Furthermore, within the context of the present invention, multiphase cosmetic antiperspirant compositions according to the invention will be understood to mean compositions which have at least 2 different phases with a phase separation and in which the phases may be arranged horizontally, that is to say one above the other, or vertically, that is to say one next to the other.

The application may take place for example using a roller ball applicator. Such rollers have a ball which is mounted in a ball bed and which can be moved by moving over a surface. In doing so, the ball picks up some of the cosmetic antiperspirant composition according to the invention that is to be distributed, and conveys this to the surface that is to be treated. As mentioned above, the packaging for the compositions according to the invention may be opaque, transparent or translucent.

Furthermore, it is also possible to apply the cosmetic antiperspirant compositions according to the invention by means of a solid stick.

However, it may also be preferred according to the invention that the cosmetic antiperspirant composition is contained on and/or in a disposable substrate selected from the group consisting of wipes, pads and dabbers. Particular preference is given to wet wipes, that is to say wet wipes which are prefabricated for the user and which are preferably packaged individually, as are well known for example from the field of glass cleaning or from the field of moist toilet paper. Such wet wipes, which advantageously may also contain preservatives, have a cosmetic antiperspirant composition according to the invention impregnated therein or applied thereto, and are preferably packaged individually. They may be used for example as a deodorant wipe, which is particularly beneficial for use when out and about. Preferred substrate materials are selected from porous flat wipes. They may consist of a fibrous or cellular flexible material which has sufficient mechanical stability and at the same time softness for use on the skin. These wipes include wipes made of woven and non-woven synthetic and natural fibres, felt, paper or foam, such as hydrophilic polyurethane foam. Deodorant or antiperspirant substrates which are preferred according to the invention may be obtained by soaking or impregnating or even by melting a cosmetic antiperspirant composition according to the invention onto a substrate.

The cosmetic antiperspirant compositions according to the invention may also include further auxiliaries. Preferably, the cosmetic antiperspirant compositions according to the invention include at least one further auxiliary selected from the group consisting of (i) emulsifiers and/or surfactants; (ii) thickeners; (iii) chelating agents; (iv) deodorant active ingredients; (v) monovalent and/or polyvalent alcohols and/or polyethylene glycols; (vi) skin-cooling active ingredients; (vii) propellants and (viii) mixtures thereof.

Suitable emulsifiers and surfactants which are preferred according to the invention are selected from anionic, cationic, non-ionic, amphoteric, in particular ampholytic and zwitterionic emulsifiers and surfactants. Surfactants are amphiphilic (bifunctional) compounds which consist of at least one hydrophobic and at least one hydrophilic moiety. The hydrophobic radical is preferably a hydrocarbon chain having 8 to 28 carbon atoms, which may be saturated or unsaturated, linear or branched. With particular preference, this C₈-C₂₈ alkyl chain is linear.

Anionic surfactants will be understood to mean surfactants having exclusively anionic charges; they include, for example, carboxyl groups, sulphonic acid groups or sulphate groups. Particularly preferred anionic surfactants are alkyl sulphates, alkyl ether sulphates, acyl glutamates and C₈₋₂₄ carboxylic acids and the salts thereof, the so-called soaps.

Cationic surfactants will be understood to mean surfactants having exclusively cationic charges; they include, for example, quaternary ammonium groups. Preference is given to cationic surfactants of the quaternary ammonium compound, esterquat and amidoamine type. Preferred quaternary ammonium compounds are ammonium halides and the imidazolium compounds known by the INCI names Quaternium-27 and Quaternium-83. Other cationic surfactants which can be used according to the invention are the quaternized protein hydrolysates. Esterquats are quaternized ester salts of fatty acids with triethanolamine, quaternized ester salts of fatty acids with diethanolalkylamines and quaternized ester salts of fatty acids with 1,2-dihydroxypropyldialkylamines.

The amphoteric surfactants are divided into ampholytic surfactants and zwitterionic surfactants. Ampholytic surfactants will be understood to mean those surface-active compounds which have both acidic (for example —COOH or —SC₃H groups) and basic hydrophilic groups (for example amino groups) and thus behave in an acidic or basic manner depending on conditions. Zwitterionic surfactants are understood by a person skilled in the art to mean surfactants which carry both a negative and a positive charge in the same molecule. Examples of preferred zwitterionic surfactants are the betaines, the N-alkyl-N,N-dimethylammonium glycinates, the N-acylaminopropyl-N,N-dimethylammonium glycinates and the 2-alkyl-3-carboxymethyl-3-hydroxyethylimidazolines having in each case 8 to 24 carbon atoms in the alkyl group. Examples of preferred ampholytic surfactants are N-alkylglycines, N-alkylaminopropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids having in each case 8 to 24 carbon atoms in the alkyl group.

The compositions according to the invention which are formulated as an emulsion, in particular as an oil-in-water emulsion, preferably include at least one non-ionic oil-in-water emulsifier having an HLB value of more than 7 to 20. These are the emulsifiers which are generally known to a person skilled in the art, as listed for example in Kirk-Othmer, “Encyclopedia of Chemical Technology,” 3rd edition, 1979, Vol. 8, pages 913-916. For ethoxylated products, the HLB value is calculated according to the formula HLB=(100−L):5, wherein L is the proportion by weight of the lipophilic groups, that is to say of the fatty alkyl or fatty acyl groups, in the ethylene oxide adducts, expressed in percent by weight. In this connection, it may be preferred according to the invention if use is also made of a water-in-oil emulsifier having an HLB value of more than 1.0 and less than/equal to 7.0. Suitable non-ionic oil-in-water emulsifiers and suitable non-ionic water-in-oil emulsifiers within the context of the present invention are described for example in the German laid-open specification DE 102006004957 A1.

In order to thicken the cosmetic antiperspirant compositions according to the invention, use is preferably made of substances selected from cellulose ethers, especially hydroxyalkyl celluloses, in particular hydroxypropyl cellulose, hydroxypropyl methyl cellulose, hydroxyethyl cellulose, carboxymethyl cellulose, cetyl hydroxyethyl cellulose, hydroxybutyl methyl cellulose, methyl hydroxyethyl cellulose, also xanthan gum, sclerotium gum, succinoglucans, polygalactomannans, in particular guar gums and locust bean gum, in particular guar gum and locust bean gum itself and the non-ionic hydroxyalkyl guar derivatives and locust bean gum derivatives, such as hydroxypropyl guar, carboxymethyl hydroxypropyl guar, hydroxypropyl methyl guar, hydroxyethyl guar and carboxymethyl guar, also pectins, agar, carrageenan, tragacanth, gum arabic, karaya gum, tara gum, gellan gum, gelatin, casein, propylene glycol alginate, alginic acids and salts thereof, in particular sodium alginate, potassium alginate and calcium alginate, also polyvinylpyrrolidones, polyvinyl alcohols, polyacrylamides, also—although less preferred—physically (for example by pregelatinization) and/or chemically modified starches, in particular hydroxypropylated starch phosphates and octenyl starch succinates and the aluminum, calcium or sodium salts thereof, also—again less preferred—acrylic acid/acrylate copolymers, acrylic acid/acrylamide copolymers, acrylic acid/vinylpyrrolidone copolymers, acrylic acid/vinylformamide copolymers and polyacrylates. Particularly preferred thickeners are selected from cellulose ethers, especially from hydroxyalkyl celluloses, in particular from hydroxypropyl cellulose, hydroxypropyl methyl cellulose, hydroxyethyl cellulose, carboxymethyl cellulose, cetyl hydroxyethyl cellulose, hydroxybutyl methyl cellulose and methyl hydroxyethyl cellulose, and mixtures thereof. Hydroxyethyl cellulose is preferably used as thickener.

In order to further aid the blocking of the efferent ducts of the sweat glands by hydrogel formation when adding at least one compound of formula (LA-I), it may be advantageous to add to the cosmetic antiperspirant compositions according to the invention at least one chelating agent selected from ethylenediaminetetraacetic acid (EDTA) and salts thereof and from nitrilotriacetic acid (NTA) and mixtures of these substances, in a total amount of 0.01 to 0.5 wt %, preferably 0.02 to 0.3 wt %, in particular 0.05 to 0.1 wt %, based on the total weight of the cosmetic antiperspirant composition according to the invention. Within the context of the present invention, however, use may also be made of chelating agents selected from the group consisting of β-alanine diacetic acid, cyclodextrin, diethylenetriamine pentamethylene phosphonic acid, sodium, potassium, calcium disodium, ammonium and triethanolamine salts of ethylenediamine tetraacetic acid (EDTA), etidronic acid, hydroxyethyl ethylenediamine tetraacetic acid (HEDTA) and the sodium salts thereof, sodium salts of nitrilotriacetic acid (NTA), diethylenetriamine pentaacetic acid, phytic acid, hydroxypropyl cyclodextrin, methyl cyclodextrin, pentasodium aminotrimethylene phosphonate, pentasodium ethylenediamine tetramethylene phosphonate, pentasodium diethylenetriamine pentaacetate, pentasodium triphosphate, potassium EDTMP, sodium EDTMP, sodium dihydroxyethylglycinate, sodium phytate, sodium polydimethylglycinophenol sulphonate, tetrahydroxyethyl ethylenediamine, tetrahydroxypropyl ethylenediamine, tetrapotassium etidronate, tetrasodium etidronate, tetrasodium iminodisuccinate, trisodium ethylenediamine disuccinate and desferrioxamine.

The antiperspirant effect of the cosmetic antiperspirant compositions according to the invention can be further increased if at least one deodorant active ingredient is included in a total amount of 0.0001 to 40 wt %, preferably 0.2 to 20 wt %, preferably 1 to 15 wt %, in particular 1.5 to 5 wt %, based on the total weight of the cosmetic antiperspirant composition according to the invention. If ethanol is used in the compositions according to the invention, in the context of the present invention this is not regarded as a deodorant active ingredient but rather as a constituent of the carrier. Deodorant active ingredients which are preferred according to the invention are selected from the group consisting of (i) silver salts; (ii) aromatic alcohols, in particular 2-benzylheptan-1-ol and mixtures of 2-benzylheptan-1-ol and phenoxyethanol; (iii) 1,2-alkanediols having 5 to 12 carbon atoms, in particular 3-(2-ethylhexyloxy)-1,2-propanediol; (iv) triethyl citrates; (v) active ingredients against exoesterases, in particular against arylsulphatase, lipase, beta-glucuronidase and cystathione-β-lyase; (vi) cationic phospholipids; (vii) odor absorbers, in particular silicates, such as montmorillonite, kaolinite, illite, beidellite, nontronite, saponite, hectorite, bentonite, smectite and talc, zeolites, zinc ricinoleate, cyclodextrins; (viii) deodorizing ion exchangers; (ix) bacteriostatic agents; (x) prebiotically acting components; and (xi) mixtures of these deodorant active ingredients.

Preferred cosmetic antiperspirant compositions according to the invention also include at least one water-soluble polyvalent C₂₋₉ alkanol having 2 to 6 hydroxyl groups and/or at least one water-soluble polyethylene glycol having 3 to 50 ethylene oxide units, and mixtures thereof. These do not encompass the aforementioned deodorant active ingredients in the form of 1,2-alkanediols. Preferred alkanols are selected from 1,2-propylene glycol, 2-methyl-1,3-propanediol, glycerol, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomeric mixtures of cis- and trans-1,4-dimethylolcyclohexane, and mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 and mixtures thereof, preference being given to PEG-3 to PEG-8.

According to another embodiment of the present invention, the cosmetic antiperspirant compositions also include at least one skin-cooling active ingredient. Skin-cooling active ingredients which are suitable according to the invention are for example menthol, isopulegol and menthol derivatives, for example menthyl lactate, menthyl glycolate, menthyl ethyl oxamate, menthyl pyrrolidone carboxylic acid, menthyl methyl ether, menthoxypropanediol, menthone glycerine acetal (9-methyl-6-(1-methylethyl)-1,4-dioxaspiro(4.5)decane-2-methanol), monomenthyl succinate, 2-hydroxymethyl-3,5,5-trimethylcyclohexanol and 5-methyl-2-(1-methylethyl)cyclohexyl-N-ethyl oxamate. As skin-cooling active ingredients, preference is given to menthol, isopulegol, menthyl lactate, menthoxypropanediol, menthyl pyrrolidone carboxylic acid and 5-methyl-2-(1-methylethyl)cyclohexyl-N-ethyl oxamate, and mixtures of these substances, in particular mixtures of menthol and menthyl lactate, menthol, menthol glycolate and menthyl lactate, menthol and menthoxypropanediol or menthol and isopulegol.

Furthermore, it may be provided that the cosmetic antiperspirant compositions according to the invention include a propellant. In this case, they are packaged as an aerosol driven by a propellant gas. Preferred propellants (propellant gases) are propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes, namely both individually and in mixtures thereof. Hydrophilic propellant gases, such as carbon dioxide for example, can also be used with advantage in the context of the present invention if the proportion of hydrophilic gases is selected to be low and lipophilic propellant gas (for example propane/butane) is present in excess. Particular preference is given to propane, n-butane, iso-butane and mixtures of these propellant gases. It has been found that the use of n-butane as the sole propellant gas may be particularly preferred according to the invention. The total amount of the propellant is 20 to 95 wt %, preferably 30 to 85 wt %, in particular 40 to 75 wt %, in each case based on the total weight of the antiperspirant consisting of the cosmetic antiperspirant composition according to the invention and the propellant. In this connection, it may also be provided that the total amount of propellant is 1 to 95 wt %, preferably 2 to 85 wt %, in particular 3 to 75 wt %, in each case based on the total weight of the antiperspirant consisting of the cosmetic antiperspirant composition according to the invention and the propellant.

According to the invention, lipophilic thickeners may also be used as auxiliaries. Preferably, the at least one antiperspirant aluminum salt is suspended in undissolved form in at least one cosmetic oil which is liquid at 20° C. and 1013 hPa. For the sake of better application, at least one lipophilic thickener may be added to this suspension as a suspending aid. Lipophilic thickeners which are preferred according to the invention are selected from hydrophobicized clay minerals and pyrogenic silicas.

In one preferred embodiment, the cosmetic antiperspirant compositions according to the invention are characterized in that they are packaged as a water-in-oil emulsion and include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %,
12 to 90 wt %, preferably 25 to 55 wt %, preferably 30 to 50 wt %, in particular 35 to 45 wt % water,
at least one emulsifier, and
at least one substance selected from the group consisting of cosmetic oils which are liquid at 20° C. and 1013 hPa, odorants and waxes.

In another preferred embodiment, the cosmetic antiperspirant compositions according to the invention are characterized in that they are packaged as an oil-in-water emulsion and include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %,
15 to 90 wt %, preferably 25 to 55 wt %, preferably 30 to 50 wt %, in particular 35 to 45 wt % water,
at least one emulsifier, and
at least one substance selected from the group consisting of cosmetic oils which are liquid at 20° C. and 1013 hPa, odorants and waxes.

Another preferred embodiment of the present invention encompasses cosmetic antiperspirant compositions according to the invention which are characterized in that they include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %,
15 to 90 wt %, preferably 25 to 80 wt %, preferably 30 to 75 wt %, in particular 40 to 60 wt % water, and
0.01 to 2 wt %, preferably 0.1 to 1 wt %, preferably 0.2 to 0.7 wt %, in particular 0.3 to 0.5 wt % of a thickener.

Another preferred embodiment of the present invention encompasses cosmetic antiperspirant compositions according to the invention which are characterized in that they include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %,
15 to 90 wt %, preferably 25 to 80 wt %, preferably 30 to 75 wt %, in particular 40 to 60 wt % water, and
0.01 to 2 wt %, preferably 0.1 to 1 wt %, preferably 0.2 to 0.7 wt %, in particular 0.3 to 0.5 wt % of a thickener,
   wherein the cosmetic antiperspirant compositions according to the invention have a dynamic viscosity in the range of 10 to 5000 mPas, preferably 100 to 1000 mPas, preferably 200 to 800 mPas, measured using a Brookfield viscosimeter, spindle RV 4, 20 s⁻¹, without Helipath, at 20° C. ambient temperature and 20° C. sample temperature.

Another preferred embodiment of the present invention encompasses cosmetic antiperspirant compositions according to the invention which are characterized in that they include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 8 wt %, preferably 0.1 to 7 wt %, preferably 0.3 to 5 wt %, more preferably 0.5 to 3 wt %, even more preferably 0.8 to 2.5 wt %, in particular 1 to 2 wt %,
15 to 90 wt %, preferably 25 to 80 wt %, preferably 30 to 75 wt %, in particular 40 to 60 wt % water, and
0.01 to 2 wt %, preferably 0.1 to 1 wt %, preferably 0.2 to 0.7 wt %, in particular 0.3 to 0.5 wt % of a thickener,
   wherein the cosmetic antiperspirant compositions according to the invention have a dynamic viscosity in the range of 1000 to 800,000 mPas, preferably 2000 to 700,000 mPas, preferably 3000 to 500,000 mPas, measured using a Brookfield viscosimeter, spindle RV 4, 20 s⁻¹, without Helipath, at 20° C. ambient temperature and 20° C. sample temperature.

In another preferred embodiment, the cosmetic antiperspirant compositions according to the invention are characterized in that they are packaged as a water-in-oil emulsion and include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %,
15 to 75 wt %, preferably 25 to 60 wt %, preferably 30 to 55 wt %, in particular 35 to 50 wt % water.

In another preferred embodiment, the cosmetic antiperspirant compositions according to the invention are characterized in that they are packaged as an oil-in-water emulsion and include, based on the total weight of the cosmetic antiperspirant composition according to the invention,
at least one antiperspirant aluminum salt in a total amount of 2 to 75 wt %, preferably 3 to 70 wt %, preferably 4 to 65 wt %, more preferably 5 to 55 wt %, even more preferably 8 to 40 wt %, in particular 10 to 30 wt %,
at least one compound of formula (LA-I) in a total amount of in total 0.05 to 20 wt %, preferably 0.1 to 15 wt %, preferably 0.3 to 10 wt %, more preferably 0.5 to 7 wt %, even more preferably 0.8 to 5 wt %, in particular 1 to 3 wt %,
15 to 90 wt %, preferably 25 to 80 wt %, preferably 30 to 75 wt %, in particular 40 to 60 wt % water, and
optionally 0.01 to 2 wt %, preferably 0.1 to 1 wt %, preferably 0.2 to 0.7 wt %, in particular 0.3 to 0.5 wt % of a thickener.

Within the context of the present invention, it may also be provided to package the cosmetic antiperspirant composition according to the invention as a two-component composition. The individual components will to this end be stored in separate containers and applied to the skin one after the other in any order. Cosmetic compositions according to the invention may therefore exist as a kit-of-parts containing, separately from one another,
a) at least one first container containing a cosmetic composition including at least one antiperspirant aluminum-containing compound, and
b) at least one second container containing a cosmetic composition including at least one compound of formula (LA-I), wherein the cosmetic composition contains less than 0.5 wt %, preferably less than 0.2 wt %, preferably less than 0.05 wt %, more preferably less than 0.005 wt %, in particular 0 wt % aluminum-containing compounds.

Within the context of the present invention, the term “aluminum-containing compounds” will be understood to mean antiperspirant aluminum salts and aluminum zirconium salts.

A second subject matter of the present invention is the use of a combination of at least one substance selected from the group consisting of cosmetic oils which are liquid at 20° C. and 1013 hPa, odorants and waxes, at least one antiperspirant aluminum salt in a total amount of 0.1 to 80 wt %, based on the total weight of the combination, and at least one compound of formula (LA-I)

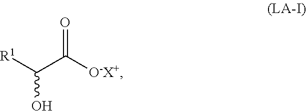

in which
R¹ is a linear or branched alkyl group having 1 to 10 carbon atoms, and
X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt,
   for reducing and/or preventing sweat. Within the context of the present invention, the term “combination” also encompasses a mixture of the at least one substance and of the antiperspirant aluminum salt with the at least one compound of formula (LA-I). With regard to the use of the aforementioned combination, what has been stated above in respect of the cosmetic antiperspirant compositions according to the invention applies mutatis mutandis.

Furthermore, a third subject matter of the present invention is a non-therapeutic cosmetic method for preventing and/or reducing the perspiration of the body, in which a cosmetic antiperspirant composition, comprising at least one substance selected from the group consisting of cosmetic oils which are liquid at 20° C. and 1013 hPa, odorants and waxes, at least one antiperspirant aluminum salt in a total amount of 0.1 to 80 wt %, based on the total weight of the cosmetic antiperspirant composition, and at least one compound of formula (LA-I), is applied to the skin, in particular to the skin of the armpits. With regard to the method according to the invention, what has been stated above in respect of the cosmetic antiperspirant compositions according to the invention applies mutatis mutandis.

However, within the context of the method according to the invention, it may also be provided that first a cosmetic composition including an antiperspirant aluminum salt is applied, and then an aluminum-salt-free cosmetic composition including at least one compound of formula (LA-I) is applied. However, it is also possible first to apply the aluminum-salt-free cosmetic composition including at least one compound of formula (LA-I) and then to use a cosmetic composition including an antiperspirant aluminum salt. The time interval between the use of the two compositions is from 10 seconds to 24 hours.

Finally, a fourth subject matter of the present invention is the use of at least one compound of formula (LA-I)

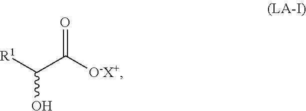

in which
R¹ is a linear or branched alkyl group having 1 to 10 carbon atoms, and
X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt,
   for enhancing the sealing of the sweat gland(s) and/or for sealing the sweat gland(s).

According to the invention, sealing of the sweat gland(s) will be understood to mean the formation of a hydrogel and/or precipitate from the at least one antiperspirant aluminum salt and the at least one compound of formula (LA-I) in the efferent duct of the sweat gland or the efferent ducts of the sweat glands. However, within the context of the present invention, sealing will also be understood to mean the aiding of the formation of a precipitate of the antiperspirant aluminum salt through the addition of the compound of formula (LA-I). By virtue of this sealing, a secretion of sweat from the efferent duct is avoided or reduced. With regard to the use according to the invention of the compound of formula (LA-I), what has been stated above in respect of the cosmetic antiperspirant compositions according to the invention applies mutatis mutandis.

Furthermore, within the context of the present invention, it is also possible to dry a liquid mixture of the at least one antiperspirant aluminum salt with the at least one compound of formula (LA-I). The drying of this mixture may be carried out for example by means of conventional drying methods, such as spray drying. The powder obtained in this way is excellent for storage and has a long storage stability.

The following examples explain the present invention, but without limiting the latter thereto:

### EXAMPLES

### 1. Hydrogel Formation

The hydrogel formation upon adding the compounds of formula (LA-Ia) or (LA-Ib) to a solution which includes an antiperspirant aluminum salt is determined as follows:

100 ml of demineralized water are placed into a glass beaker and then 35 ml of a dilution of the solution indicated in Table 1 is added. The solutions of Table 1 are to this end diluted in a glass beaker to an aluminum salt content of 1.25 wt %. The pH value of the solution in the glass beaker is then adjusted to pH 6.5 to 6.6 by adding a 2% strength sodium bicarbonate solution, while stirring. Immediately after the pH value has been adjusted, the turbidity of the solution is measured using a Methrom Optrode 6.1115.000 at a wavelength of 574 nm (greenish yellow) in mV (resolution 0.1 mV). The higher the turbidity of the solution, the lower the measurement value obtained in mV. Each measurement is carried out twice, and the mean value is formed therefrom. The turbidity is an indication of the potential of the mixture to form a hydrogel.

TABLE 1
Solutions for the turbidity measurement (figures in wt %)

| | V-I | E-I* | E-II* |
|---|---|---|---|
| Aluminum chlorohydrate | 10 | 10 | 10 |
| Compound of formula (LA-Ia) | — | 2 | — |
| Compound of formula (LA-Ib) | — | — | 2 |

*according to the invention

The difference Δturbidity between the turbidity of the comparison solution V-I and the turbidity of example E-I or E-II according to the invention (Δturbidity [mV]=turbidity V-I−turbidity E-I or E-II) is shown in Table 2 below.

TABLE 2
Difference Δturbidity in mV

| | Δturbidity in mV |
|---|---|
| E-I | 25 |
| E-II | 77 |

The addition of the compound of formula (LA-Ia) or (LA-Ib) leads, at a pH of 6.5 to 6.6, to a significantly higher turbidity or hydrogel formation in the samples E-I and E-II according to the invention than in the comparison sample V-I. In addition, the ammonium salt of lactic acid according to the compound of formula (LA-Ib) leads to a much higher turbidity in comparison to the potassium salt of lactic acid according to compound (LA-Ia). By adding the compounds of formula (LA-Ia) or (LA-Ib), the hydrogel formation is increased and an effective sealing of the efferent ducts of the sweat glands is ensured, the ammonium salt of lactic acid (LA-Ib) having a greater efficacy than the potassium salt of lactic acid.

### 2. Formulations

The compound of formula (LA-I) used in the following examples is preferably the potassium salt of lactic acid (formula LA-Ia), the ammonium salt of lactic acid (formula LA-Ib) and the mixture thereof:

Cosmetic Antiperspirant Compositions According to the Invention (Amounts in Wt %)

| | 1 | 2 | 3 | 4 | 5 | 6 |
|---|---|---|---|---|---|---|
| Hydrogenated castor oil | — | — | — | 1.5 | 1.5 | 1.5 |
| Stearyl alcohol | 24.0 | 24.0 | 24.0 | 18 | 18 | 18 |
| Novata AB | — | — | — | 4 | 4 | 4 |
| Powder of ACH and com- | 15.0 | 22.0 | 20.0 | 11.6 | 15.6 | 12.6 |
| pound of formula (LA-I) | | | | | | |
| (spray-dried, contains | | | | | | |
| 10 wt % (LA-I)) | | | | | | |
| Al—Zr-pentachlorohydrex | 7.00 | — | — | 6.00 | — | — |
| Gly | | | | | | |
| PPG-14 butyl ether | 10.0 | 10.0 | 10.0 | 15.3 | 15.3 | 15.3 |
| Hydrogenated castor oil | 3.0 | 3.0 | 3.0 | — | — | — |
| (e.g. Cutina HR) | | | | | | |
| Myristyl myristate | 1.5 | 1.5 | 1.5 | — | — | — |
| DL-menthol | 0.2 | 0.2 | 0.2 | — | — | — |
| Eucalyptol | 0.2 | 0.2 | 0.2 | — | — | — |
| Anethol | 0.2 | 0.2 | 0.2 | — | — | — |
| Silica dimethyl silylate | 1.4 | 1.4 | 1.4 | — | — | — |
| Silica | 0.3 | 0.3 | 0.3 | — | — | — |
| Talc | — | — | — | 3 | 3 | 3 |
| Emulgin B1 | — | — | — | 3 | 3 | 3 |
| Fragrance | 2.0 | 2.0 | 2.0 | 1 | 1 | 1 |
| Cyclomethicone (at least 95 | ad | ad | ad | ad | ad | ad |
| wt % cyclopentasiloxane) | 100 | 100 | 100 | 100 | 100 | 100 |

Cosmetic Antiperspirant Compositions According to the Invention in the Form of an Oil-in-Water Emulsion (Amounts in Wt %)

| | | |
|---|---|---|
| 7 | 8 | 9 |

| | | | |
|---|---|---|---|
| Cutina ® AGS | 2.5 | 2.5 | 2.5 |
| Cutina ® FS45 | 3.5 | 3.5 | 3.5 |
| Eumulgin ® B2 | 0.8 | 0.8 | 0.8 |
| Eumulgin ® B3 | 0.8 | 0.8 | 0.8 |
| Diisopropyl adipate | 6.0 | 6.0 | 6.0 |
| Novata ® AB | 4.0 | 4.0 | 4.0 |
| Cutina ® CP | 5.0 | 5.0 | 5.0 |
| Cutina ® HR | 4.0 | 4.0 | 4.0 |
| Kesterwachs K62 | 5.0 | 5.0 | 5.0 |
| Locron ® L (ACH solution 50% | 40 | 40 | 30 |
| strength) | | | |
| Talc Pharma G | 10 | 10 | 10 |
| Fragrance | 1.2 | 1.2 | 1.2 |
| 2-Benzylheptan-1-ol | — | 0.3 | 0.3 |
| Sensiva SC 50 | 0.6 | 0.6 | 0.6 |
| Compound of formula (LA-I) | 2.0 | 3.0 | 3.0 |
| 1,2-Propanediol | 10 | 10 | 10 |
| Water, demineralized | ad 100 | ad 100 | ad 100 |

Cosmetic Antiperspirant Compositions According to the Invention in the Form of Microemulsions (Amounts in Wt %)

| | 10 | 11 | 12 | 13 |
|---|---|---|---|---|
| Plantaren ® 1200 | 1.7 | 1.7 | — | — |
| Plantaren ® 2000 | 1.1 | 1.4 | 2.4 | 2.4 |
| Glycerol monooleate | 0.71 | 0.71 | — | — |
| Dioctyl ether | 4.0 | 4.0 | 0.090 | 0.090 |
| Octyldodecanol | 1.0 | 1.0 | 0.020 | 0.020 |
| Fragrance oil | 1.0 | 1.0 | 1.0 | 1.0 |
| Aluminum chlorohydrate | 8.0 | 5.0 | 5.0 | 10.0 |
| 1,2-Propylene glycol | 5.0 | 5.0 | — | — |
| Glycerol | — | — | 5.0 | 5.0 |
| 2-Benzylheptan-1-ol | 0.50 | — | — | — |
| Triethyl citrate | — | 0.50 | 0.50 | 0.50 |
| Triclosan | 0.10 | — | — | — |
| Compound of formula (LA-I) | 1.0 | 2.0 | 2.5 | 2.5 |
| Water | ad 100 | ad 100 | ad 100 | ad 100 |

Cosmetic Antiperspirant Compositions According to the Invention in the Form of Roll-Ons (Amounts in Wt %)

| | 14 | 15 | 16 | 17 |
|---|---|---|---|---|
| Ethanol 96% strength (DEP | 30 | 30 | 28 | 28 |
| denatured) | | | | |
| Mergital ® CS 11 | 2.0 | 2.0 | — | — |
| Eumulgin ® B3 | 2.0 | 2.0 | 2.0 | 2.0 |
| Emulgin ® B1 | — | — | 2.0 | 2.0 |
| Aluminum chlorohydrate 50% | — | 20 | — | 16 |
| (Locron L) | | | | |
| Al-Zr-pentachlorohydrex Gly | 20 | — | 16 | — |
| Hydroxyethyl cellulose | 0.50 | 0.50 | 0.30 | 0.30 |
| Compound of formula (LA-I) | 2.5 | 0.50 | 2.0 | 1.5 |
| EDTA | — | — | — | 0.050 |
| Cocamidopropyl PG-dimonium | 0.20 | — | — | — |
| chloride phosphate | | | | |
| Fragrance oil | 0.80 | 0.80 | 1.0 | 1.0 |
| Water | ad 100 | ad 100 | ad 100 | ad 100 |

### Antiperspirant Wipes

### Examples No. 18 to 21

For the embodiment according to the invention as an antiperspirant wipe, in each case 75 g of example emulsions 14 or 15 per square meter or in each case 75 g of example compositions 10 or 11 were applied to a monolayer substrate composed of 100% viscose having a surface weight of 50 g/m², and the latter was cut into wipes of suitable size and packaged in sachets.

Cosmetic Antiperspirant Compositions According to the Invention in the Form of a Water-in-Oil Emulsion (Amounts in Wt %)

| | 22 | 23 | 24 |
|---|---|---|---|
| Aluminum chlorohydrate 50% in water | 35.6 | 35.6 | 20.0 |
| (Locron L) | | | |
| 1,2-Propylene glycol | 13.0 | 13.0 | 13.0 |
| Cyclohexasiloxane | 6.00 | 6.00 | 6.00 |
| Finsolv TN | 8.00 | 8.00 | 8.00 |
| Abil EM 90 | 1.20 | 1.20 | 1.20 |
| Polyethylene wax (MW = 500 g/mol, | 10.0 | 10.0 | 10.0 |
| Smp = 83 to 91° C.) | | | |
| Polyalphaolefin wax (MW = 1800 g/mol, | 0.100 | 0.100 | 0.100 |
| Smp = 41° C.) | | | |
| Compound of formula (LA-I) | 2.00 | 0.500 | 0.500 |
| EDTA | — | 0.0500 | 0.0500 |
| Water | 25.0 | 25.0 | ad 100 |
| Fragrance | 1.00 | 1.00 | 1.00 |

Cosmetic Antiperspirant Compositions According to the Invention (Amounts in Wt %)

| | 25 | 26 |
|---|---|---|
| Cyclopentasiloxane | 14.0 | 14.0 |
| Abil EM 97 | 3.00 | 3.00 |
| Ethanol 96% | 10.0 | 10.0 |
| Aluminum chlorohydrate 50% in water (Locron L) | 40.0 | 40.0 |
| 1,2-Propylene glycol | 20.3 | 20.3 |
| Water | 11.6 | 11.6 |
| Compound of formula (LA-I) | 2.00 | 0.500 |
| EDTA | — | 0.0750 |
| Fragrance | 1.00 | 1.00 |

Cosmetic Antiperspirant Compositions According to the Invention (Amounts in Wt %, Based on the Total Weight of the Propellant-Free Composition)

| | 27 | 28 | 29 | 30 |
|---|---|---|---|---|
| Aluminum chlorohydrate (ACH) | 28.6 | 14.29 | 32.11 | 28.57 |
| Bentone 38 V CG | 5.00 | 3.93 | 4.00 | 5.00 |
| Propylene carbonate | 1.50 | 0.71 | 1.50 | 1.80 |
| Fragrance | 7.14 | 6.50 | 5.00 | 6.50 |
| 2-Ethylhexyl palmitate | — | 73.57 | — | — |
| Abil K 4 | 48.4 | — | — | — |
| Isopropyl myristate | 7.37 | — | 10.00 | 19.22 |
| Triethyl citrate | — | — | 10.5 | 19.2 |
| C10-C13 isoalkane | — | — | 35.39 | 19.21 |
| Compound of formula (LA-I) | 2.00 | 1.00 | 1.50 | 0.500 |

Example compositions 27 to 30 were filled into an aluminum spray can, which was optionally coated with epoxy phenolic lacquer, in a weight ratio of propellant (butane/propane/isobutene mixture) to suspension of respectively 80:20 or 85:15 or 60:40 or 90:10.

Cosmetic Antiperspirant Compositions According to the Invention (Amounts in Wt %, Based on the Total Weight of the Propellant-Free Composition)

| | 31 | 32 | 33 |
|---|---|---|---|
| Aluminum chlorohydrate (ACH) | 33.0 | 33.0 | 33.0 |
| C₁₀-C₁₃ isoalkane | 8.90 | 8.90 | 8.90 |
| Dow Corning ES-5227 DM | 1.40 | 1.40 | 1.40 |
| Isoceteth-20 | 0.500 | 0.500 | 0.500 |
| Dimethicone | 4.20 | 4.20 | 4.20 |
| Isopropyl myristate | 9.00 | 9.00 | 9.00 |
| 1,2-Propanediol | 7.00 | 25.0 | 25.0 |
| Phenoxyethanol | 0.500 | 0.500 | 0.500 |
| Fragrance | 2.50 | 2.50 | 2.50 |
| Compound of formula (PP-I) | 2.00 | 0.500 | 1.50 |
| L-Menthol | 0.400 | 0.300 | — |
| trans-Anethol | — | 0.300 | — |
| Eucalyptol | — | 0.300 | — |
| Water | ad 100 | ad 100 | ad 100 |

Example compositions 31 to 33 were filled into an aluminum spray can, which was optionally coated with epoxy phenolic lacquer, in a weight ratio of propellant (butane/propane/isobutene mixture) to suspension of respectively 80:20 or 85:15 or 60:40 or 90:10.

Cosmetic Antiperspirant Compositions According to the Invention in the Form of O/W Emulsions (Amounts in Wt %)

| | 34 | 35 | 36 |
|---|---|---|---|
| Aluminum chlorohydrate (ACH) | 13.0 | 13.0 | 13.0 |
| Brij S 2 | 2.50 | 2.50 | 2.50 |
| Brij S 721 | 1.50 | 1.50 | 1.50 |
| Fragrance | 1.10 | 1.10 | 1.10 |
| Arlamol E | 0.500 | 0.500 | 0.500 |
| Bisabolol | 0.100 | 0.100 | 0.100 |
| Dry Flo PC | 0.100 | 0.100 | 0.100 |
| Compound of formula (PP-I) | 2.00 | 3.00 | 1.00 |
| Dow Corning 2501 Cosmetic Wax | 0.100 | 0.100 | 0.100 |
| Tocopheryl acetate | 0.100 | 0.100 | 0.100 |
| Water | ad 100 | ad 100 | ad 100 |

Cosmetic Antiperspirant Compositions According to the Invention (Amounts in Wt %, Based on the Total Weight of the Propellant-Free Composition)

| | 37 | 38 | 39 | 40 |
|---|---|---|---|---|
| Aluminum chlorohydrate (ACH) | 33.0 | 33.0 | 33.0 | 33.0 |
| Cyclomethicone | 12.0 | 9.40 | — | — |
| C10-C13 isoalkane | — | — | 9.40 | 8.90 |
| Dow Corning ES-5227 DM | — | 1.40 | 1.40 | 1.40 |
| Abil EM 90 | 3.00 | — | — | — |
| Brij IC 20 | — | — | — | 0.500 |
| Dimethicone | — | 4.20 | 4.20 | 4.20 |
| Isopropyl myristate | 9.00 | 9.00 | 9.00 | 9.00 |
| Compound of formula (PP-I) | 2.50 | 1.00 | 3.00 | 0.500 |
| 1,2-Propanediol | 7.00 | 7.00 | 7.00 | 7.00 |
| Phenoxyethanol | 0.500 | 0.500 | 0.500 | 0.500 |
| Fragrance | 2.50 | 2.50 | 2.50 | 2.50 |
| Water | ad 100 | ad 100 | ad 100 | ad 100 |

Example compositions 37 to 40 were filled into an aluminum spray can, which was optionally coated with epoxy phenolic lacquer, in a weight ratio of propellant (butane/propane/isobutene mixture) to suspension of respectively 80:20 or 85:15 or 60:40 or 90:10.

Cosmetic Antiperspirant Compositions According to the Invention in the Form of W/O Emulsions (Amounts in Wt %)

| | 41 | 42 | 43 | 44 |
|---|---|---|---|---|
| Aluminum chlorohydrate 50% in | 62.5 | 62.5 | 60.0 | 58.0 |
| water (Locron L) | | | | |
| Propylene glycol | 5.00 | 5.00 | 7.50 | 9.50 |
| C12-C15 alkyl benzoate | 8.04 | 8.04 | 8.04 | 8.04 |
| Dimethicone 2 cst | 6.43 | 6.43 | 6.43 | 6.43 |
| Dimethicone 5 cst | 1.57 | 1.57 | 1.57 | 1.57 |
| Polyethylene | 10.2 | 11.7 | 9.70 | 12.2 |
| Abil EM 90 | 0.998 | 0.998 | 0.998 | 0.998 |
| Abil EM 97 | 1.22 | 1.22 | 1.22 | 1.22 |
| Compound of formula (PP-I) | 2.50 | 1.00 | 3.00 | 0.500 |
| Synthetic wax | 0.100 | 0.100 | 0.100 | 0.100 |
| Fragrance | 1.50 | 1.50 | 1.20 | 1.50 |

The following commercial products were used:

| | | Supplier/ |
|---|---|---|
| Commercial product | INCI | Manufacturer |
| Abil EM 90 | CETYL PEG/PPG-10/1 | Evonik |
| | Dimethicone | |
| Abil EM 97 | Bis-PEG/PPG-14/14 | Evonik |
| | Dimethicone, | |
| | Cyclomethicone | |
| Abil K 4 | Cyclomethicone | Goldschmidt |
| Arlamol E | PPG-15 Stearyl ether | Croda |
| Bentone 38 V CG | Disteardimonium Hectorite | Elementis |
| | | Specialities |
| Brij IC 20 | Isoceteth-20 | Croda |
| Brij S 2 | Steareth-2 | Croda |
| Brij S 721 | Steareth-21 | Croda |
| Cutina ® CP | Cetyl Palmitate | BASF |
| Cutina ® FS45 | Palmitic Acid, Stearic Acid | BASF |
| Cutina ® HR | Hydrogenated Castor Oil | BASF |
| Dow Corning ® 245 | Cyclopentasiloxane | Dow Corning |
| Dow Corning ® 2501 | Bis-PEG-18 Methyl ether | Dow Corning |
| | dimethyl silane | |
| Dow Corning ES-5227 | Dimethicone, PEG/PPG- | Dow Corning |
| DM | 18/18 Dimethicone in a | |
| | weight ratio 3:1 | |
| Dry Flo PC | Aluminum Starch | National Starch |
| | Octenylsuccinate | |
| Eumulgin ® B1 | Ceteareth-12 | BASF |
| Eumulgin ® B2 | Ceteareth-20 | BASF |
| Eumulgin ® B3 | Ceteareth-30 | BASF |
| Kesterwachs K62 | Cetearyl Behenate | Koster Keunen |
| Finsolv TN | C12-15 Alkyl Benzoate | Innospec |
| Locron L (AS = 50%) | Aluminum Chlorohydrate | Clariant |
| Mergital ® CS 11 | Ceteareth-11 | BASF |
| Novata ® AB | Cocoglycerides (melting | BASF |
| | point 30-32° C.) | |
| Plantaren ® 1200 | LAURYL GLUCOSIDE, | BASF |
| | approx. 50% AS | |
| Plantaren ® 2000 | DECYL GLUCOSIDE, | BASF |
| | approx. 50% AS | |
| Sensiva ® SC 50 | 2-Ethylhexylglycerin ether | Schülke & Mayr |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic antiperspirant composition, comprising:
a) at least one substance selected from the group consisting of cosmetic oils which are liquid at 20° C. and 1013 hPa, odorants, and waxes;
b) at least one antiperspirant aluminum salt in a total amount of 0.1 to 80 wt %, based on the total weight of the cosmetic antiperspirant composition, and
c) at least one compound of formula (LA-I), 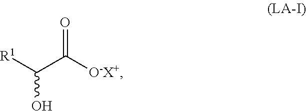
in which
R¹ is a linear or branched alkyl group having 1 to 10 carbon atoms, and
X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt.

2. The cosmetic antiperspirant composition according to claim 1, wherein the at least one substance (a) is an odorant and comprises 0.00001 to 10% by weight based on the total weight of the cosmetic antiperspirant composition.

3. The cosmetic antiperspirant composition according to claim 1, wherein the at least one substance (a) is a wax and comprises 0.01 to 20% by weight based on the total weight of the cosmetic antiperspirant composition.

4. The cosmetic antiperspirant composition according claim 1, wherein the at least one substance (a) is a cosmetic oil which is liquid at 20° C. and 1013 hPa, said cosmetic oil being selected from the group consisting of volatile cyclic silicone oils, volatile nonsilicone oils, nonvolatile silicone oils, nonvolatile nonsilicone oils, and mixtures thereof.

5. The cosmetic antiperspirant composition of claim 4, wherein the cosmetic oil includes a volatile cyclic silicone oils selected from the group consisting of cyclotrisiloxane, cyclotetrasiloxane, cyclopentasiloxane, and cyclohexasiloxane, and linear silicone oils having 2 to 10 siloxane units.

6. The cosmetic antiperspirant composition of claim 5, wherein the linear silicone oils having 2 of 10 siloxane units are selected from the group consisting of hexamethyldisiloxane, octamethyltrisiloxane, and decamethyltetrasiloxane.

7. The cosmetic antiperspirant composition of claim 4, wherein the cosmetic oil includes a volatile nonsilicone oils selected from the group consisting of liquid paraffin oils and isoparaffin oils.

8. The cosmetic antiperspirant composition of claim 7, wherein the cosmetic oil is selected from the group consisting of isodecane, isoundecane, isododecane, isotridecane, isotetradecane, isopentadecane, isohexadecane, and isoeicosane.

9. The cosmetic antiperspirant composition of claim 4, wherein the cosmetic oil is a high molecular linear polyalkylsiloxane.

10. The cosmetic antiperspirant agent of claim 4, wherein the cosmetic oil is selected from the group consisting of esters of linear or branched, saturated or unsaturated C₂₋₃₀ fatty alcohols with linear or branched, saturated or unsaturated C₂₋₃₀ fatty acids, which may be hydroxylated, the C₈-C₂₂ fatty alcohol esters of monovalent or polyvalent C₂-C₇ hydroxycarboxylic acids, the triethyl citrates, the branched saturated or unsaturated C₆₋₃₀ fatty alcohols, the mono-, di-, and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide with monohydric or polyhydric C₃₋₂₂ alkanols, which may optionally be esterified, the symmetrical, asymmetrical, or cyclic esters of carbonic acid with fatty alcohols, the esters of dimers of unsaturated C₁₂₋₂₂ fatty acids with monohydric linear, branched, and cyclic C₂₋₁₈ alkanols or C₂₋₆ alkanols, the benzoic acid esters of linear or branched C₈₋₂₂ alkanols, the synthetic hydrocarbons, the alicyclic hydrocarbons.

11. The cosmetic antiperspirant composition according to claim 1, wherein the antiperspirant aluminum salt comprises 1 to 40% by weight of the cosmetic antiperspirant composition.

12. The cosmetic antiperspirant composition according to claim 1, wherein antiperspirant aluminum salt is selected from the group of water-soluble astringent inorganic salts of aluminum, water-soluble astringent organic salts of aluminum, water-soluble astringent inorganic aluminum-zirconium salts, water-soluble astringent organic aluminum-zirconium salts, and mixtures thereof.

13. The cosmetic antiperspirant composition according to claim 1, wherein the antiperspirant is selected from the group consisting of aluminum chlorohydrate, aluminum sesquichlorohydrate, aluminum dichlorohydrate, aluminum hydroxide, potassium aluminum sulfate, aluminum bromohydrate, aluminum chloride, aluminum sulfate, aluminum chlorohydrex propylene glycol, aluminum chlorohydrex polyethylene glycol, aluminum-propylene glycol complexes, aluminum sesquichlorohydrex propylene glycol, aluminum sesquichlorohydrex polyethylene glycol, aluminum dichlorohydrex propylene glycol, aluminum dichlorohydrex polyethylene glycol, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate, aluminum zirconium trichlorohydrate, aluminum zirconium tetrachlorohydrate, aluminum zirconium pentachlorohydrate, aluminum zirconium octachlorohydrate, aluminum-zirconium/propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine, and mixtures thereof.

14. The cosmetic antiperspirant composition according to claim 1, wherein the compound of formula (LA-I) comprises 0.05 to 20 wt % of the cosmetic antiperspirant composition.

15. The cosmetic antiperspirant composition according to claim 1, wherein the cosmetic antiperspirant composition includes at least one compound of formula (LA-Ia) and/or (LA-Ib)

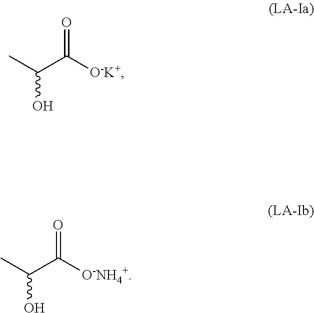

16. The cosmetic antiperspirant composition according to claim 1, wherein the cosmetic antiperspirant composition has a weight ratio of the antiperspirant aluminum salt to the compound of formula (M-I) of 40:1 to 19:1.

17. The cosmetic antiperspirant composition according to claim 1, wherein the cosmetic antiperspirant composition has a weight ratio of the antiperspirant aluminum salt to the compound of formula (M-I) of 10:1 to 13:1.

18. The cosmetic antiperspirant composition according to claim 1, wherein the cosmetic antiperspirant composition includes free water in a total quantity of 15 to 96% by weight based on the total weight of the cosmetic antiperspirant composition.

19. The cosmetic antiperspirant composition according to claim 1, wherein the cosmetic antiperspirant composition includes ethanol in a total quantity of 1 to 50% by weight based on the total weight of the cosmetic antiperspirant composition.

20. A method for enhancing the sealing of the sweat gland(s) and/or sealing the sweat gland(s), comprising:
applying to the skin surface of a subject at least one compound of formula (LA-I) 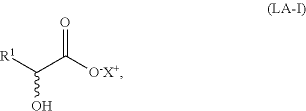
in which
R¹ is a linear or branched alkyl group having 1 to 10 carbon atoms, and
X⁺ is Li⁺, K⁺, NH₄⁺, ½ Mg²⁺, ½ Ca²⁺, ½ Mn²⁺, ½ Zn²⁺, ⅓ Al³⁺, ¼ Zr⁴⁺ or at least one antiperspirant aluminum salt.
